(19) United States
(12) Patent Application Publication
(10) Pub. No.: US 2005/0101673 A1
(43) Pub. Date: May 12, 2005

(54) Use of orally available prostacyclin derivatives for the production of a pharmaceutical agent for treating diseases that are associated with bone marrow edemas

(75) Inventors: Cornelia Norden, Berlin (DE); Nicolas Aigner, Wien (AT)

Correspondence Address: MILLEN, WHITE, ZELANO & BRANIGAN, P.C., 2200 CLARENDON BLVD., SUITE 1400, ARLINGTON, VA, 22201, US

(73) Assignee: Schering AG, Berlin (DE)

(21) Appl. No.: 10/455,820

(22) Filed: Jun. 6, 2003

Related U.S. Application Data
(60) Provisional application No. 60/391,618, filed on Jun. 27, 2002.

(30) Foreign Application Priority Data
Jun. 6, 2002 (DE) 10225551.2

(51) Int. Cl. A61K 31/557

(52) U.S. Cl. 514/573

(57) ABSTRACT

The invention relates to the use of orally available prostacyclin derivatives for the production of a pharmaceutical agent for treating diseases that are associated with bone marrow edemas.

The invention relates to the subject that is indicated in the claims, i.e., the use of orally available prostacyclin derivatives for the production of a pharmaceutical agent for treating diseases that are associated with bone marrow edemas.

For osteonecrosis and recently also for osteoarthritis in the knee, a connection between the presence of a bone marrow edema and pain is assumed (Felson et al., Ann Intern Med, 2001, 134(7); 541-593).

Osteonecrosis (syn.: aseptic necrosis, avascular necrosis, ischemic necrosis) is a common disease. In this case, pain is the indicating sign. In about two thirds of the patients, this pain occurs at rest. Osteonecrosis is often described as a transient process. When the course proceeds spontaneously, in most cases what happens is a complete healing after 6 to 12 months. In this period, however, the patient is burdened by intense pain that is often not affected even by a commonly used pain therapy. The incidence rates of the progression of an early osteonecrosis up to the end stage with pronounced bone necroses are not known. Osteonecrosis is responsible for more than 10% of all joint replacement operations performed annually.

Osteoarthritis in the knee is a common disease that attacks about 11-15% of those over 65 years of age and is the most frequent cause of physical handicap in older humans. The main reason for physical limitations and visits to physicians is knee pain. Osteoarthritis is defined as primarily non-inflammatory joint destruction that begins with cartilage degeneration in adults, later also affects common joint structures and progresses irreversibly. The overall course is generally progressive, but alternating in phases in terms of clinical acuity. In the final stage, knee replacement is necessary.

Osteonecrosis and osteoarthritis are most often manifested on hip and knee joints, but also on shoulder joints, wrists and ankles.

While in the treatment of osteonecrosis, surgical bone marrow decompression (perforation) with all its disadvantages such as in-patient stay and surgical intervention is performed as a means for alleviating pain, the treatment of osteoarthritis now exists only in symptomatic pain treatment and is inadequately effective. It usually comprises the permanent prescription of analgesics and/or antiphlogistic agents, combined with physiotherapy and weight reduction as well as optionally the intra-articular injection of anesthetics, corticoids or “cartilage-building/-replacing” substances. X-ray excitation radiation and acupuncture are also prescribed. Therapy of arthrosis is also limited here to date to a symptomatic treatment.

The established therapies are unsatisfactory both for osteonecrosis and for osteoarthritis because of the limitation to symptomatic treatment. In particular, the continued existence of dolorogenic mechanisms requires a long-term treatment that is associated with additional risks and costs. A causal treatment of pain with permanent freedom from pain is therefore especially desirable for osteoarthritis.

It is known that alleviation of pain in osteonecrosis can be achieved by a five-day intravenous infusion therapy with Ilomedin® (iloprost-trometamol) (Aigner et al., J Bone Joint Surg, 2001, 83(6): 855-858). The freedom from pain promptly reached with the reduction of bone marrow edema does not free the physician from close controls over an extended period, however, since the freedom from pain that is achieved masks a possible progression in a necrotic stage of the bone. Beyond the freedom from pain, treatment of necrotic lesions with the purpose of obtaining or restoring healthy bone tissue is therefore desirable.

The object was therefore to find a treatment of diseases that are associated with bone marrow edemas, especially osteonecrosis and osteoarthritis, that is not limited to only symptomatic treatment, i.e., combating pain, but rather also restores the structural integrity of the bone, and in addition to make available a pharmaceutical agent that can be taken over an extended period by the patient himself without the support of the physician.

It has now been found, surprisingly enough, that orally available prostacyclin derivatives, especially the cyclodextrin clathrates of the prostacyclin derivatives of general formula I 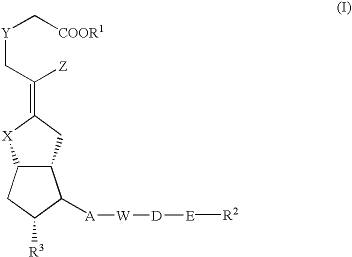
in which
R¹ means a hydrogen atom or a C₁-C₁₀-alkyl radical,
A means a —CH₂—CH₂— group, a trans —CH═CH— group or a —C≡C— group,
W means a free hydroxymethylene group, or a hydroxymethylene group that
   is functionally modified to form a hydroxy group,
   whereby the hydroxy group is in α- or β-position,
X, Y, independently of one another, mean a —CH₂— group or an oxygen atom,
Z means a hydrogen atom or a cyano group,
D means a straight-chain or branched, saturated C₁-C₅-alkylene group,
E means a —C≡C— group or a direct bond,
R² means a straight-chain or branched, saturated C₁-C₇-alkyl group,
R³ means a free or functionally modified hydroxy group,
and all optically active forms, racemates, diastereomers, diastereomer mixtures, clathrates thereof,
and if R¹ has the meaning of a hydrogen atom, the salts thereof with physiologically compatible bases, achieve this object.

A 28-day treatment of patients with painful bone marrow edema by iloprost β-cyclodextrin clathrate (iloprost oral) reduced not only the bone marrow edema but also restored the structural integrity, i.e., necrotic areas and lesions were healed. The alleviation of pain and the improved articulation could be observed even two months after the end of treatment. Treatment with orally available prostacyclin derivatives, especially with cyclodextrin clathrates of the prostacyclin derivatives of formula I, thus represents a novel therapy that not only suppresses the symptoms, i.e., the pain, but combats the causes of osteonecrosis and osteoarthritis and for the first time can also be clinical, studied independently.

This finding is all the more surprising in that earlier studies, such as, e.g., the placebo-controlled studies for treatment of severe ischemia of the legs, did not show iloprost β-cyclodextrin clathrate to have any effectiveness (Eur J Vasc Endovasc Surg., 2000, 20(4): 358-362). The effectiveness of the intravenous treatment with iloprost-trometamol, which is approved for treatment of the serious ischemia of the legs, thus could not be reproduced.

It is consequently not possible to predict that iloprost β-cyclodextrin clathrate will have an advantageous action in every case based on knowledge regarding intravenous iloprost-trometamol.

As alkyl group R¹, straight-chain or branched alkyl groups with 1-10 carbon atoms can be considered, such as, for example, methyl, ethyl, propyl, 2-methylethyl, n-butyl, 1-methylpropyl, 2-methylpropyl, 1,1-dimethylethyl(tert-butyl), hexyl, heptyl, octyl, nonyl or decyl.

Alkyl group R¹ can also be substituted. As substituents, for example, fluorine, chlorine or bromine atoms, phenyl, dimethylamino, diethylamino, methoxy, and ethoxy can be mentioned.

As alkyl group R¹, alkyl groups with 1-4 carbon atoms are preferred.

Alkyl group R² is straight-chain or branched; by way of example, reference can be made to the chain length that corresponds to radicals that are mentioned for R¹, and alkyl group R² has 1-7 carbon atoms, preferably 1-3 carbon atoms, especially preferably 1-2.

The hydroxy groups in R³ and W can be functionally modified. Thus, an esterification or etherification is meant. The thus obtained ethers and/or acyl radicals are radicals that are known to one skilled in the art.

Ether radicals are, e.g., tetrahydropyranyl, tetrahydrofuranyl, methoxymethyl, methoxyethyl, ethoxyethyl, silyl ether, such as, e.g., trimethylsilyl, dimethyl-tert-butylsilyl, or triphenylsilyl.

Acyl radicals can be, for example, acetyl, propionyl, butyryl, or benzoyl.

X preferably has the meaning of a CH₂ group.

Z preferably means a hydrogen atom.

As alkylene group D, straight-chain or branched saturated alkylene groups with 1-5 carbon atoms are considered. For example, methylene, ethylene, propylene, 1-methylethylene, butylene, 1-methyl-propylene, 2-methylpropylene, pentylene, 1-methylbutylene, and 1 -ethylethylene can be mentioned. A branched alkylene group with 3 carbon atoms is preferred.

If R¹ means a hydrogen atom, the compounds of formula I can also be present as salts of physiologically compatible bases.

Both inorganic and organic bases are suitable for salt formation. For example, alkali hydroxides such as sodium or potassium hydroxide; alkaline-earth hydroxides such as calcium hydroxide, or ammonia; and amines such as ethanolamine, diethanolamine, triethanolamine, N-methylglucamine, morpholine, or tris-(hydroxymethyl)methylamine can be mentioned.

Prostacyclin derivatives are orally available as defined by the invention at the time when they can be used according to suitable formulation for oral administration.

In addition, especially α- or β-cyclodextrin clathrates or hydroxylated forms thereof are suitable.

Especially suitable for the treatment of diseases that are associated with bone marrow edemas, especially for the treatment of osteonecroses and osteoarthritis, are also the following known prostacyclin derivatives of general formula I that are formulated for oral administration: cicaprost (5-{(E)-(1S,5S,6S,7R)-7-hydroxy-6-[(3S,4S)-3-hyroxy-4-methyl-1,6-nonadiinyl]-bicyclo[3.3.0]-oct-3-ylidene}-3-oxapentanoic acid), epoprostenol ((5Z,9α,13E,15S)-6,9-epoxy-11,15-dihydroxy-prosta-5,13-diene-1-acid), beraprost (EP 0 084 856), ciprostene, taprostene, naxaprostene, CS 570, SC-39902, FCE-22509, OP 41483 and RS 93427.

The β-cyclodextrin clathrate of iloprost, (5- {(1S,5S,6R,7R)-7-hydroxy-6-[(E)-(3R,4RS)-3-hydroxy-4-methyl-oct-1-en-6-inyl]-bicyclo[3.3.0]octane-3-ylidene}pentanoic acid)-β-cyclodextrin clathrate, is especially suitable. Within a treatment of 28 days, a significant improvement of the pain that is immediately noticeable to the patients and that is associated with a significant reduction of the bone marrow edema and a healing of necroses takes place.

The use of iloprost-β-cyclodextrin clathrate for the production of a pharmaceutical agent for treating osteonecrosis and osteoarthritis is therefore a preferred subject of the invention.

Iloprost is a prostacyclin analog that is known from EP 11 591 and can be produced by methods disclosed therein. The nomenclature name for iloprost is (5-{(1S,5S,6R,7R)-7-hydroxy-6-[(E)-(3R,4RS)-3-hydroxy-4-methyl-oct-1-en-6-inyl]-bicyclo[3.3.0]octane-3-ylidene}pentanoic acid).

Iloprost-β-cyclodextrin-clathrate is known from EP 259468 and can be produced according to methods that are disclosed there.

It is known from EP 1 016 408 that prostanoids are C—C chemokine-production inhibitors. For the bone marrow edema, the mechanisms that are disclosed there are not relevant according to present knowledge.

With respect to conventional methods for treating osteonecroses and osteoarthritis, avoiding a cost-intensive in-patient stay, cutting down on surgical interventions (perforation, especially for osteonecroses), reducing the necessity for implantation of artificial joints, avoiding costs for treating side effects of conventional pain medications, reducing the morbidity, shortening the disability time, improving the quality of life and reducing the number of visits to the doctor are significant advantages.

In particular, the oral administration makes possible a longer-term medication with a causal therapy stock that the patients can take largely independently of any medical support.

The suitable dose range for the treatment of osteonecrosis and osteoarthritis is between 50 μg/day and 350 μg/day, preferably between 100 μg/day and 300 μg/day or between 150 μg/day and 350 μg/day, whereby these amounts can be distributed with several individual dosages. A healing of the bone necroses can be achieved within a few weeks.

### DESCRIPTION OF THE FIGURES

FIG. 1: Significant abatement of the resting pain under oral iloprost treatment measured by means of a 100 mm visual analog scale with end points 0=no pain and 100=intolerable pain. The alleviation of pain is already considerable after 3 days of treatment. Almost complete freedom from pain is achieved after a 28-day treatment and persists over a period of 2 months without therapy.

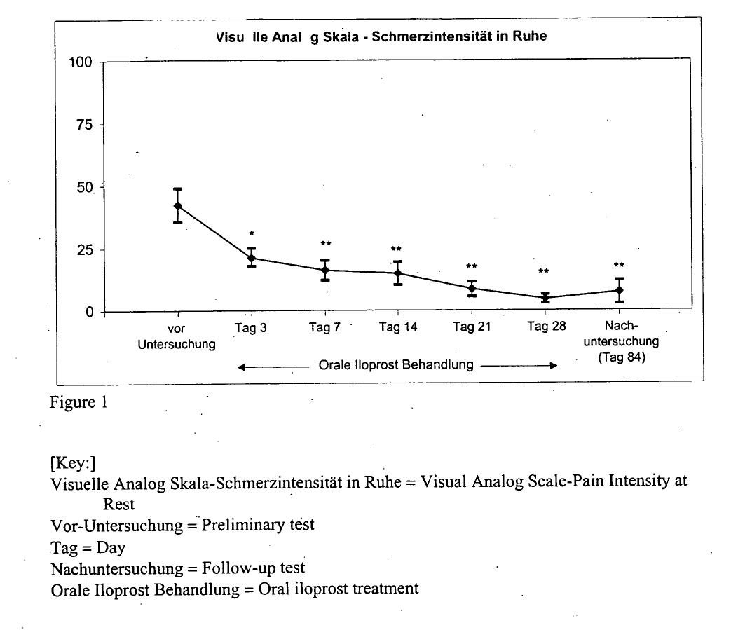

Data as average values±SEM. p-Values as a comparison of the corresponding times with the starting value, with *p<0.05 and **p<0.001 (ANOVA).

FIG. 2: Significant abatement in pain after defined stress measured by means of a 100 mm visual analog scale with end points 0=no pain and 100=intolerable pain.

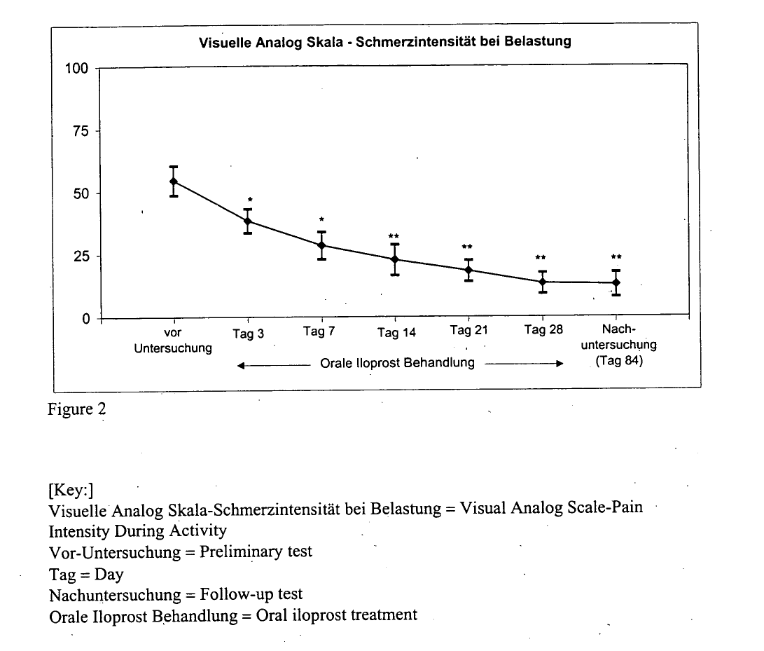

The alleviation of pain is already considerable after 3 days of treatment. Almost complete freedom from pain is achieved after a 28-day treatment and persists over a period of 2 months without therapy.

Data as average values±SEM. p-Values as a comparison of the corresponding times with the starting value, with *p<0.05 and **p<0.001 (ANOVA).

FIG. 3: Significant increase of the knee activity score according to Larson (maximum score 100) after a 14-day treatment. The improvement persists over a period of 2 months without therapy.

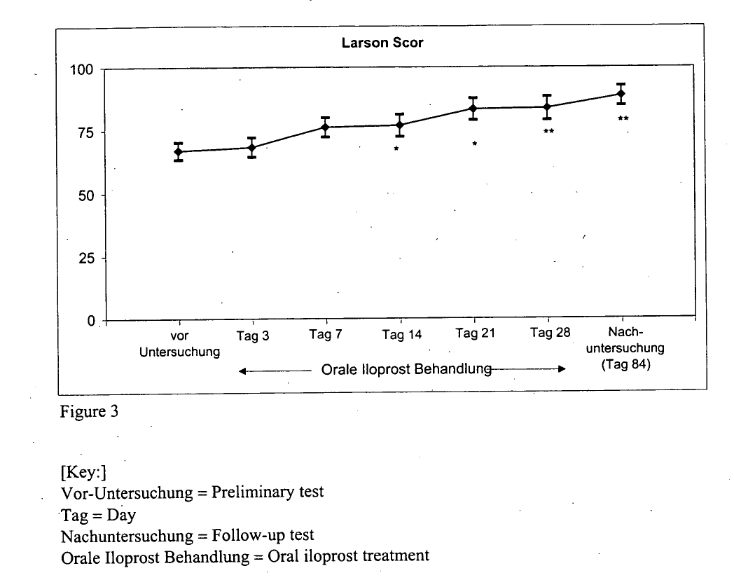

Data as average values±SEM. p-Values as a comparison of the corresponding times with the starting value, with *p<0.05 and **p<0.001 (ANOVA).

FIG. 4: Case example of a patient with complete degeneration of a diffused bone marrow edema in the early stages of osteonecrosis. The control-MRI study was carried out 2 months after the 28-day treatment with oral iloprost was completed.

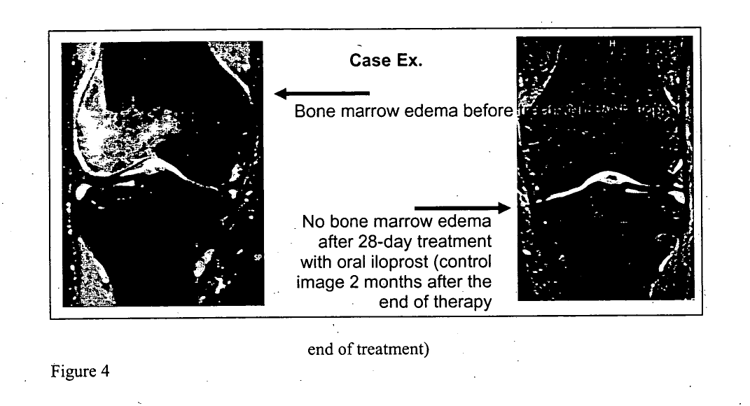

FIG. 5: Same pronounced abatement in resting pain when administered in a double-blind trial of oral iloprost treatment and Tramadol® treatment measured by means of a 100 mm visual analog scale with end points 0=no pain and 100=intolerable pain. Almost complete freedom from pain is achieved after 28 days of treatment and persists over a period of 2 months without therapy.

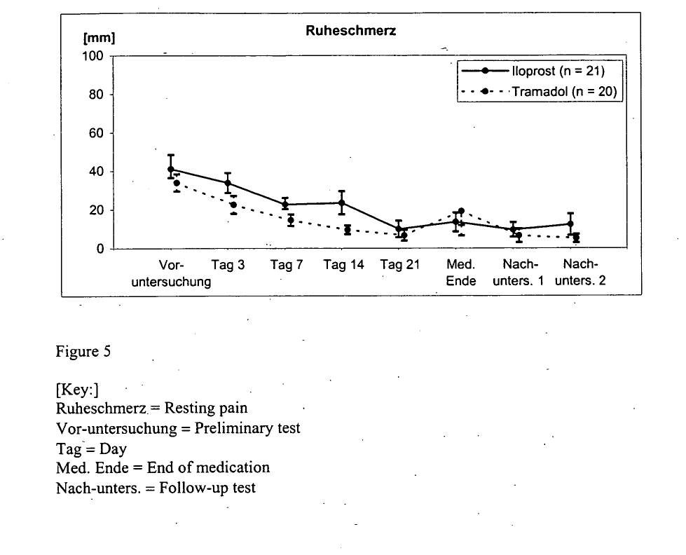

Data as average values±SEM. p-Values for comparing the two treatments confirm their equivalence (Hodges-Lehmann-Schätzer and Wilcoxon Test).

FIG. 6: Same pronounced abatement in activity-related pain under double-blind administration of oral iloprost treatment and Tramadol® treatment measured by means of a 100 mm visual analog scale with the end points 0=no pain and 100=intolerable pain. A considerable reduction in pain is achieved after 28 days of treatment and persists over a period of 2 months without therapy.

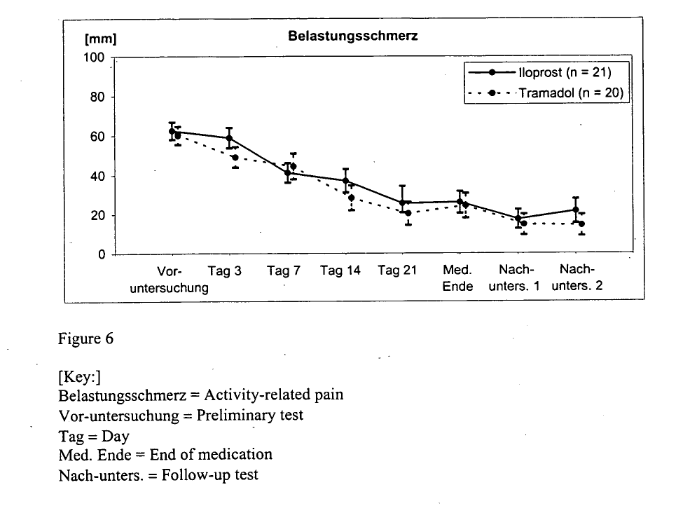

Data as average values±SEM. p-Values for comparing the two treatments confirm their equivalence (Hodges-Lehmann-Schätzer and Wilcoxon Test).

FIG. 7: Same pronounced increase in the knee activity score according to Larson (maximum score 100) under double-blind administration of oral iloprost treatment and Tramadol® treatment. The improvement persists over a period of 2 months without therapy.

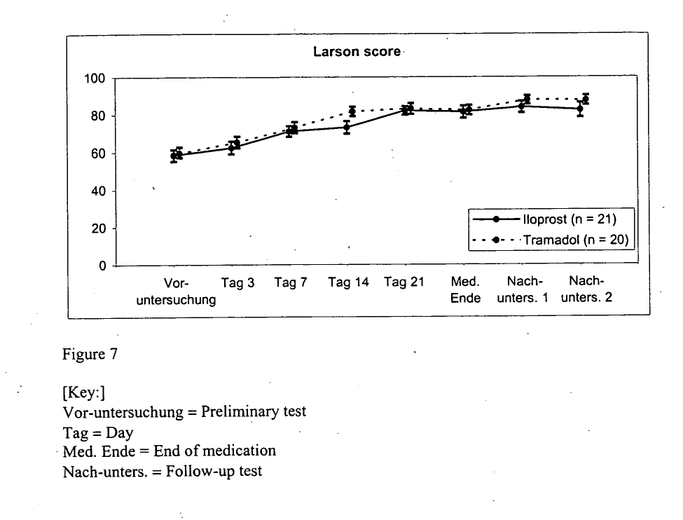

Data as average values±SEM. p-Values for comparing the two treatments confirm their equivalence (Hodges-Lehmann-Schätzer and Wilcoxon Test).

FIG. 8: Oral iloprost produced a complete degeneration of the bone marrow edema in at least one affected bone in 53% of patients, while this was the case in only 19% of the patients under Tramadol®.

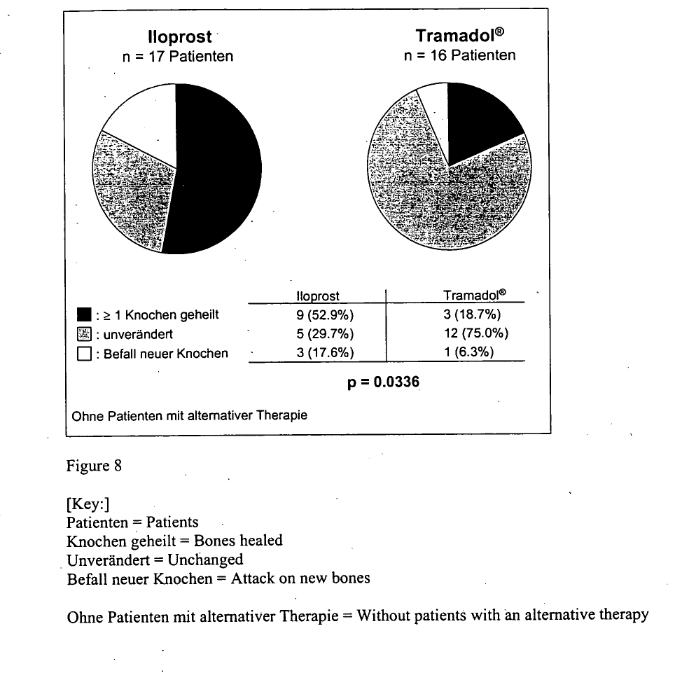

This difference was statistically significant (p=0.0336, Fisher's Exact Test)

### EXPERIMENTS

Pilot Studies

For the action of oral iloprost on the bone marrow edema and pain, an open pilot study with 19 patients with osteonecrosis of the knee was performed in the 1. Orthopädische Abteilung des Orthopadischen Spital Wien-Speising [1^{st} Orthopedic Department of the Orthopedic Hospital of Vienna-Speising], Prim. Dr. F. Landsiedl/Dr. N. Aigner, in the period from March to November 2001.

Before the beginning of the study, all patients gave their written consent to participate in the study after they were informed extensively orally and in writing on the type, purpose and course of the study.

Patients of both sexes with resting pain because of an osteonecrosis were included in the study if the following criteria were met:
Age between 19 and 60 years,
Bone marrow edema found by means of MRT according to specified criteria,
Normal x-ray finding without signs of osteoarthritis and no subchondral attack of the bone.

The treatment was carried out with iloprost capsules that were packaged and characterized according to valid GLP rules. Each capsule contained 50 μg of iloprost.

During the first three days of treatment, iloprost was administered in a dosage of 3×50 μg of iloprost during the day, which starting on day 4 could be increased individually up to a maximum dose of 3×100 μg of iloprost during the day:
To increase the success of the therapy (alleviation of pain), this dosage could be carried out both by increasing the individual dose (maximum: 3×100 μg of iloprost during the day) and by shortening the intake-free interval (minimum: 2 hours, i.e., 6×50 μg of iloprost during the day).
In the case of poor tolerance, the dosage could always be reduced to a minimum dose of 2×50 μg of iloprost during the day.

The treatment period was one month. Previously administered pain-relieving medications should be discontinued before the beginning of the study.

All medications for the treatment of accompanying diseases were able to be maintained at the usual dosages.

The clinical action of the treatment was determined based on the following parameters:
Evaluation of the intensity of the resting pain and the activity-related pain by the patient based on a 100 mm scale, whereby 0 means “no pain,” and 100 means “intolerable pain.” The stress was defined as climbing and descending a specified set of stairs.
Evaluation of the functional condition of the knee by means of Larson scores.
Evaluation of the bone marrow edema by means of MRT.

As safety parameters, undesirable events and side effects were detected. Known side effects of iloprost are symptoms such as headache, reddening of the face, nausea, vomiting and diarrhea.

The course of the study was presented as follows:

| | | | | | | | Follow- | |
|---|---|---|---|---|---|---|---|---|
| | | Treatment | | | | | up Test | |
| | | 1 | 2 | 3 | 4 | 5 | 6 | 7 |
| | Preliminary | Day | Day | Day | Day | Day | Day | Day |
| Visit | Test | 3 | 7 | 14 | 21 | 28 | 56 | 84 |
| Declaration of consent | ● | | | | | | | |
| Inclusion and exclusion criteria | ● | | | | | | | |
| Case history | ● | | | | | | | |
| Physical test | ● | | | | | | | |
| Pregnancy test | ● | | | | | ● | ● | ● |
| Polling for side effects | | ● | ● | ● | ● | ● | | |
| Action parameters: | | | | | | | | |
| Larson Score | ● | ● | ● | ● | ● | ● | ● | ● |
| Patient evaluation of the pain | ● | ● | ● | ● | ● | ● | ● | ● |
| intensity | | | | | | | | |
| MRT | ● | | | | | | | ● |
| X-ray image of the affected | ● | | | | | | | |
| knee¹⁾ | | | | | | | | |

¹⁾The study could take place over six weeks.

As a result of a 28-day treatment with oral iloprost, freedom from pain at rest and during activity was almost achieved in patients with osteonecrosis, the majority of whom, before the beginning of the study, had unsuccessfully taken a number of other medication combinations over several weeks. The action on the pain could already be detected after a 3-day treatment and persisted 2 months after the end of the treatment. With the reduction of pain, a complete degeneration of bone marrow edema was noted in MR control images in 44% of the patients.

Double-Blind Comparison Studies

To confirm the action of oral iloprost on the bone marrow edema and the pain, a random, double-blind comparison study was performed on the opiate Tramadol® in 41 patients with osteonecrosis or osteoarthritis of the knee in the period from March 2001 to November 2002 according to the GCP guidelines.

Before the beginning of the study, all patients gave their written consent to participate in the study after they were informed extensively orally and in writing on the type, purpose and course of the study.

Patients of both sexes with resting pain because of an osteonecrosis or osteoarthritis were included in the study if the following criteria were met:
Age between 19 and 60 years,
Bone marrow edema found by means of MRT according to specified criteria.

The treatment was carried out either with iloprost capsules or with outwardly indistinguishable Tramadol capsules that were packaged and characterized according to valid GLP rules. Each capsule contained either 50 μg of iloprost or 50 mg of Tramadol®.

During the first three days of treatment, the study medication was administered in a dosage of 3×1 capsules during the day, which starting on day 4 could be increased individually to increase the success of the therapy (alleviation of pain) up to a maximum dose of 3×2 capsules during the day. In the case of poor tolerance, the dosage could always be reduced to a minimum dose of 2×1 capsules during the day.

The treatment period was one month, followed by a follow-up observation period of two months during which no treatment was given. Previously administered pain-relieving medications should be discontinued before the beginning of the study.

All medications for the treatment of accompanying diseases were able to be maintained at the usual dosages.

The clinical action of the treatment was determined based on the following parameters:
Evaluation of the intensity of the resting pain and the activity-related pain by the patient based on a 100 mm scale, whereby 0 means “no pain,” and 100 means “intolerable pain.”
Evaluation of the functional condition of the knee by means of Larson scores.
Evaluation of the bone marrow edema by means of MRT.

As safety parameters, undesirable events and side effects were detected. Known side effects of iloprost are symptoms such as headache, reddening of the face, nausea, vomiting and diarrhea.

The course of the study was presented as follows:

| | | | | | | | | | | Follow- | |
|---|---|---|---|---|---|---|---|---|---|---|---|
| | | | | | | | | | | up | |
| | Preliminary | | Treatment¹⁾ | | | | | Alternative | | Test¹⁾ | |
| | Test | | 1 | 2 | 3 | 4 | 5²⁾ | Therapy | | 6⁴⁾ | 7⁵⁾ |
| | | Base- | Day | Day | Day | Day | Day | Day | Day | Day | Day |
| Visit | Screening | line | 3 | 7 | 14 | 21 | 28 | 1-4 | 5³⁾ | 56 | 84 |
| Declaration of consent | ● | | | | | | | | | | |
| Inclusion and exclusion | ● | ● | | | | | | | | | |
| criteria | | | | | | | | | | | |
| Demography, general | ● | | | | | | | | | | |
| and specific anamnesis | | | | | | | | | | | |
| Employment status | ● | | | | | | | | | | |
| Work disability | ● | ● | ● | ● | ● | ● | ● | ● | ● | ● | ● |
| Physical test | ● | | | | | | ● | | ● | | ● |
| Pregnancy test | ● | | | | | | ● | | ● | ●⁶⁾ | ● |
| Randomization | | ● | | | | | | | | | |
| Dispensing of research | | ● | | ● | ● | ● | | | | | |
| medication | | | | | | | | | | | |
| Withdrawal of unused | | | | ● | ● | ● | ● | | | | |
| medication | | | | | | | | | | | |
| Dose/dosage adjustment, | | | ● | ● | ● | ● | | ● | ● | | |
| if necessary | | | | | | | | | | | |
| Polling for side effects | | | ● | ● | ● | ● | ● | | | ● | ● |
| Blood pressure, pulse | ● | | ● | ● | ● | ● | ● | ● | ● | ● | ● |
| Blood samples | ● | | | | | | ● | | ● | | ● |
| (hematology, clinical | | | | | | | | | | | |
| chemistry), urinalysis | | | | | | | | | | | |
| Preliminary and | ● | | ● | ● | ● | ● | ● | ● | ● | ● | ● |
| accompanying treatment | | | | | | | | | | | |
| Action parameters: | | | | | | | | | | | |
| Larson score | | ● | ● | ● | ● | ● | ● | | ● | ● | ● |
| Patient evaluation of the | | ● | ● | ● | ● | ● | ● | ● | ● | ● | ● |
| pain intensity | | | | | | | | | | | |
| MRT | ● | | | | | | | | | | ● |
| X-ray image of the affected | ●⁷⁾ | | | | | | | | | | |
| knee | | | | | | | | | | | |

¹⁾Visits outside of the planned time guide could be carried out both during treatment and during the follow-up test.
²⁾End of research medication or early stopping of the research medication.
³⁾End of alternative therapy or early stopping of alternative therapy.
⁴⁾Visit 6 took place on day 56 or after 4 treatment-free weeks.
⁵⁾Visit 7 took place on day 84 or after 8 treatment-free weeks.
⁶⁾Urine-strip test.
⁷⁾The study can take place within 6 weeks of the baseline.

In patients with osteonecrosis or osteoarthritis, oral iloprost produced a degeneration of the bone marrow edema in 53% of patients, while this was the case in only 19% of the patients under Tramadol®. This difference was statistically significant (p=0.0336, Fisher's Exact Test). This effect coincided with a significant alleviation of pain as also shown in the pilot study. Moreover, only with iloprost therapy was a reduction in subchrondral lesions noted and thus a reference to the restoration of structural integrity was provided.

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing and in the examples, all temperatures are set forth uncorrected in degrees Celsius, and all parts and percentages are by weight, unless otherwise indicated.

The entire disclosure[s] of all applications, patents and publications, cited herein and of corresponding German Application No. 102 25 551.2, filed Jun. 6, 2002, and U.S. Provisional Application Ser. No. 60/391,618, filed Jun. 27, 2002, are incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. Use of orally available prostacyclin derivatives of general formula I 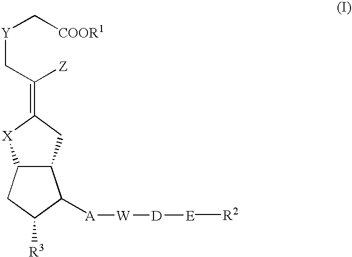
in which
R¹ means a hydrogen atom or a C₁-C₁₀-alkyl radical,
A means a —CH₂—CH₂— group, a trans —CH=CH— group or a —C≡C— group,
W means a free hydroxymethylene group, or a hydroxymethylene group that
is functionally modified to form a hydroxy group,
whereby the hydroxy group is in α- or β-position,
X, Y, independently of one another, mean a —CH₂— group or an oxygen atom,
Z means a hydrogen atom or a cyano group,
D means a straight-chain or branched, saturated C₁-C₅-alkylene group,
E means a —C≡C— group or a direct bond,
R² means a straight-chain or branched, saturated C₁-C₇-alkyl group,
R³ means a free or functionally modified hydroxy group,
and all optically active forms, racemates, diastereomers, diastereomer mixtures, clathrates thereof,
and if R¹ has the meaning of a hydrogen atom, the salts thereof with physiologically compatible bases,
for the production of a pharmaceutical agent for the treatment of diseases that are associated with bone marrow edemas.

2. Use according to claim 1, characterized in that the prostacyclin derivatives of formula I are present as cyclodextrin clathrate.

3. Use of orally available prostacyclin derivatives according to claim 1 for the production of a pharmaceutical agent for the treatment of osteonecroses.

4. Use of orally available prostacyclin derivatives according to claim 1 for the production of a pharmaceutical agent for the treatment of osteoarthritis.

5. Use of orally available iloprost (5-{(1S,5S,6R,7R)-7-hydroxy-6-[(E)-(3R,4RS)-3-hydroxy-4-methyl-oct-1-en-6-inyl]-bicyclo[3.3.0]octane-3-ylidene}pentanoic acid), cicaprost (5-{(E)-(1S,5S,6S,7R)-7-hydroxy-6-[(3S,4S)-3-hydroxy-4-methyl- 1,6-nonadiinyl]-bicyclo[3.3.0]-oct-3-ylidene}-3-oxapentanoic acid), epoprostenol ((5Z,9═,13E,15S)-6,9-epoxy-11,15-dihydroxy-prosta-5,13-dienoic-1-acid), beraprost (TRK 100), ciprostene, taprostene, naxaprostene, CS 570, SC-39902, FCE-22509, OP 41483 or RS 93427 according to claim 1 for the production of a pharmaceutical agent for the treatment of diseases that are associated with bone marrow edemas.

6. Use of orally available iloprost (5-{(1S,5S,6R,7R)-7-hydroxy-6-[(E)-(3R,4RS)-3-hydroxy-4-methyl-oct-1-en-6-inyl]-bicyclo[3.3.0]octane-3-ylidene}pentanoic acid), cicaprost (5-{(E)-(1S,5S,6S,7R)-7-hydroxy-6-[(3S,4S)-3-hydroxy-4-methyl- 1,6-nonadiinyl]-bicyclo[3.3.0]-oct-3-ylidene} -3-oxapentanoic acid), epoprostenol ((5Z,9α,13E,15S)-6,9-epoxy-11,15-dihydroxy-prosta-5,13-dienoic-1-acid), beraprost (TRK 100), ciprostene, taprostene, naxaprostene, CS 570, SC-39902, FCE-22509, OP 41483 or RS 93427 according to claim 2 for the production of a pharmaceutical agent for the treatment of osteonecroses.

7. Use of orally available iloprost (5-{(1S,5S,6R,7R)-7-hydroxy-6-[(E)-(3R,4RS)-3-hydroxy-4-methyl-oct-1-en-6-inyl]-bicyclo[3.3.0]octane-3-ylidene}pentanoic acid), cicaprost (5-{(E)-(1S,5S,6S,7R)-7-hydroxy-6-[(3S,4S)-3-hydroxy-4-methyl-1,6-nonadiinyl]-bicyclo[3.3.0]-oct-3-ylidene}-3-oxapentanoic acid), epoprostenol ((5Z,9α,13E,15S)-6,9-epoxy-11,15-dihydroxy-prosta-5,13-dienoic-1-acid), beraprost (TRK 100), ciprostene, taprostene, naxaprostene, CS 570, SC-39902, FCE-22509, OP 41483 or RS 93427 according to claim 3 for the production of a pharmaceutical agent for the treatment of osteoarthritis.

8. Use of iloprost-β-cyclodextrin-clathrate according to claim 1 for the production of a pharmaceutical agent for the treatment of diseases that are associated with bone marrow edemas.

9. Use of iloprost-β-cyclodextrin-clathrate according to claim 2 for the production of a pharmaceutical agent for treating osteonecrosis.

10. Use of iloprost-β-cyclodextrin-clathrate according to claim 3 for the production of a pharmaceutical agent for the treatment of osteoarthritis.
